(19) United States
(12) Patent Application Publication
(10) Pub. No.: US 2024/0044798 A1
(43) Pub. Date: Feb. 8, 2024

(54) FLOW CELL FOR ANALYSIS OF NUCLEIC ACID AND DEVICE FOR ANALYSIS OF NUCLEIC ACID

(71) Applicant: HITACHI HIGH-TECH CORPORATION, Tokyo (JP)

(72) Inventors: Noriko EBINE, Tokyo (JP); Terumi TAMURA, Tokyo (JP); Masatoshi NARAHARA, Tokyo (JP)

(21) Appl. No.: 18/266,309

(22) PCT Filed: Dec. 21, 2020
(86) PCT No.: PCT/JP2020/047668

§ 371 Date: Jun. 9, 2023

(51) Int. Cl. G01N 21/64

(52) U.S. Cl. CPC G01N 21/6486

(57) ABSTRACT

Embodied is a flow cell that is for analysis of nucleic acid and that is capable of inhibiting complication of data processing without a reduction in throughput. The flow cell for analysis of nucleic acid comprises: a flow path formation body that has a flow path into which a nucleic acid sample flows; and a flow path formation body support member that has a region in which nucleic acid in a nucleic acid sample flowing into the flow path is to be adsorbed. The flow path formation body support member has the nucleic acid adsorption region in which nucleic acid is to be adsorbed, and a fluorescent particle adsorption region which is separated from the nucleic acid adsorption region, and in which fluorescent particles are to be adsorbed. In the fluorescent particle adsorption region, a blocking substance that can be specifically adsorbed to the fluorescent particle adsorption region is adsorbed.

### TECHNICAL FIELD

The present invention relates to a flow cell for analysis of nucleic acid and a device for analysis of nucleic acid.

### BACKGROUND ART

As a device for analyzing the base sequence of DNA, a DNA sequencer is known. The DNA sequencer is a device that denatures DNA fragments into a single-stranded state, uses the single-stranded fragments as a template to sequentially extend a fluorescence-labeled nucleic acid one base by one base, and sequentially analyzes the base sequence of DNA by fluorescence observation using a microscope.

To perform the analysis, a plate of which a part or all is formed of a transparent material, for example, a flow cell where a flow path is provided in a glass plate is prepared, and a plurality of colonies of clones of the denatured single-stranded DNA fragments are produced in the flow path. A nucleotide to which a fluorescent dye capable of identifying four types of nucleotides (A, T, G, C) forming DNA is added is caused to flow in the flow path.

Here, a functional group that inhibits the extension of the next base is added to the nucleotide. After identifying the fluorescent dye incorporated by the extension reaction, a new reagent is caused to flow to eliminate the functional group that inhibits the extension. In the flow path of the flow cell, a plurality of colonies of single-stranded DNA fragments having a size of several micrometers are provided, and by repeating a cycle of the extension reaction and the elimination reaction described above, the process of renaturing the single-stranded DNA fragments into a double-stranded state is observed with a fluorescence microscope to sequentially read the DNA base sequence.

In a genetic test using the next-generation sequencer, the presence of a certain number of specimen failures and undeterminable cases is reported. The genetic test includes multiple stages of specimen collection to determination including specimen collection and storage, pre-treatment, sequencing, data analysis, result review, and determination. To investigate causes of the undeterminable cases, it is necessary to ensure the device operation of the next-generation sequencer alone.

PTL 1 discloses a quality evaluation method regarding the entire genetic test using a reference gene. The reference gene is measured by the sequencer, information output from the sequencer is analyzed, and for example, the accuracy of each base in a gene sequence or the degree to which clusters of each gene are close to each other that is obtained as a result of the analysis is an index of the genetic test.

PTL 2 describes a device used for excitation light irradiation and inspection of a fluorescent light detection unit in the configuration of the sequencer. The device includes a material that emits known fluorescence with a known photon energy, and is used for excitation light irradiation and adjustment and inspection of a fluorescence detection unit in the sequencer.

PTL 2 also describes that, even when the shape of the material that emits known excitation light with a known photon energy is small fluorescent particles, excitation light irradiation and adjustment and inspection of a fluorescence detection unit can be performed.

PTL 3 describes that, to prevent a nucleic acid sample from removing from a sample-fixing layer, a blocking layer that covers a region other than a region where the nucleic acid sample or a carrier is bound to the sample-fixing layer is provided.

### CITATION LIST

### Patent Literature

PTL 1: JP2019-080501A
PTL 2: US2018/0195961A
PTL 3: JP2012-132778A

### SUMMARY OF INVENTION

### Technical Problem

In the method described in PTL 1, it is necessary to arrange the reference gene in a measurement area of the sequencer. In the measurement area, the area used for genes to be inspected is reduced, and there is a problem in that the device throughput decreases.

In the method described in PTL 2, when the shape of the material that emits known excitation light with a known photon energy is fluorescent particles, in a well-known example, an epoxy, a polymer, or other materials may be embedded therein. Therefore, an adjacent material of the fluorescent particles is a material different from a reagent. When genes to be inspected are measured, an adjacent material of the genes is a reagent. Irradiated light is also incident on an adjacent member of the fluorescent material. Therefore, due to a difference in optical characteristics (for example, transmittance or reflectance) of the adjacent material of the reference fluorescent material, for example, correction of signal intensity is required for the acquired image, and there is a problem in that data processing becomes complicated.

In the method described in PTL 3, it is not possible to separately arrange a sample to be inspected and two types of carriers as other materials such that only the sample is arranged in one section 1 and only the other materials are arranged in a section 2. Therefore, the material that emits excitation light with a known photon energy is also arranged in a region used for genes to be inspected, and it is not possible to solve the problem that the area used for genes to be inspected is reduced and the device throughput decreases.

An object of the invention is to implement a flow cell for analysis of nucleic acid and a device for analysis of nucleic acid capable of inhibiting complication of data processing without a reduction in throughput.

### Solution to Problem

To achieve the object, the invention is configured as follows.

A flow cell for analysis of nucleic acid includes: a flow path formation body including a flow path into which a nucleic acid sample flows; and a flow path formation body support member including a region where a nucleic acid in the nucleic acid sample flowing into the flow path is adsorbed, in which the flow path formation body support member includes a nucleic acid adsorption region where the nucleic acid is adsorbed, and a fluorescent particle adsorption region that is separated from the nucleic acid adsorption region and where fluorescent particles are adsorbed, and a blocking substance to be specifically adsorbed on the fluorescent particle adsorption region is adsorbed on the fluorescent particle adsorption region.

A device for analysis of nucleic acid includes a flow cell for analysis of nucleic acid, an excitation light source, a fluorescence detection unit, a liquid supply unit, and a data processing control unit configured to perform operation control and fluorescent image analysis, in which the flow cell for analysis of nucleic acid includes a flow path formation body including a flow path into which a nucleic acid sample flows, and a flow path formation body support member including a region where a nucleic acid in the nucleic acid sample flowing into the flow path is adsorbed, the flow path formation body support member includes a nucleic acid adsorption region where the nucleic acid is adsorbed, and a fluorescent particle adsorption region that is separated from the nucleic acid adsorption region and where fluorescent particles are adsorbed, and a blocking substance to be specifically adsorbed on the fluorescent particle adsorption region is adsorbed on the fluorescent particle adsorption region.

### Advantageous Effects of Invention

According to the invention, it is possible to implement a flow cell for nucleic acid analysis and a device for analysis of nucleic acid capable of inhibiting complication of data processing without a reduction in throughput.

### BRIEF DESCRIPTION OF DRAWINGS

FIG. 1A is a diagram illustrating a flow cell for analysis of nucleic acid according to a first embodiment of the invention.

FIG. 1B is a cross-sectional view taken along line A-A of FIG. 1A.

FIG. 1C is a cross-sectional view taken along line B-B of FIG. 1A.

FIG. 2 is a diagram illustrating an example of a method of manufacturing the flow cell for analysis of nucleic acid according to the first embodiment of the invention.

FIG. 3 is a diagram illustrating an example of the method of manufacturing the flow cell for analysis of nucleic acid according to the first embodiment of the invention.

FIG. 4A is a diagram illustrating an example of the method of manufacturing the flow cell for analysis of nucleic acid according to the first embodiment of the invention.

FIG. 4B is a cross-sectional view taken along line C-C of FIG. 4A.

FIG. 5 is a diagram illustrating an example of the method of manufacturing the flow cell for analysis of nucleic acid according to the first embodiment of the invention.

FIG. 6A is a diagram illustrating an example of the method of manufacturing the flow cell for analysis of nucleic acid according to the first embodiment of the invention.

FIG. 6B is a cross-sectional view taken along line D-D of FIG. 6A.

FIG. 7 is a diagram illustrating an example of the method of manufacturing the flow cell for analysis of nucleic acid according to the first embodiment of the invention.

FIG. 8 is a diagram illustrating an example of the method of manufacturing the flow cell for analysis of nucleic acid according to the first embodiment of the invention.

FIG. 9 is a diagram illustrating an example of the method of manufacturing the flow cell for analysis of nucleic acid according to the first embodiment of the invention.

FIG. 10 is a diagram illustrating an example of the method of manufacturing the flow cell for analysis of nucleic acid according to the first embodiment of the invention.

FIG. 11 is a diagram illustrating an example of the method of manufacturing the flow cell for analysis of nucleic acid according to the first embodiment of the invention.

FIG. 12 is a diagram illustrating an example of the method of manufacturing the flow cell for analysis of nucleic acid according to the first embodiment of the invention.

FIG. 13 is a diagram illustrating a configuration example of a device for analysis of nucleic acid according to a second embodiment of the present invention.

FIG. 14 is a diagram illustrating an example of a control system of the device for analysis of nucleic acid according to the second embodiment of the invention.

FIG. 15 is a diagram illustrating an example of a control flow of the device for analysis of nucleic acid according to the second embodiment of the invention.

FIG. 16 is a diagram illustrating an example of the control flow of the device for analysis of nucleic acid according to the second embodiment of the invention.

FIG. 17 is a diagram illustrating an example of the control flow of the device for analysis of nucleic acid according to the second embodiment of the invention.

FIG. 18 is a diagram illustrating an example of the control flow of the device for analysis of nucleic acid according to the second embodiment of the invention.

### DESCRIPTION OF EMBODIMENTS

As a result of thorough investigation, the inventors of the invention completed: as a device used for excitation light irradiation and inspection of a fluorescence detection unit in a sequencer, a flow cell for analysis of nucleic acid where correction for adjacent materials are unnecessary for a measurement result of fluorescent particles without a reduction in device throughput and fluorescent particles can be arranged in a section different from that of genes to be inspected; and a device for analysis of nucleic acid where the above-described flow cell for analysis of nucleic acid is mounted.

The invention provides a flow cell for measuring a fluorescent material in a flow path, in which the flow path includes a region where only a sample is measured and a region where fluorescent particles different from the measurement sample are present, and a blocking layer to which the measurement sample is not attached is laminated in the region where only the fluorescent particles are present.

The invention is a flow cell where fluorescent particles are fixed at a position that is not used for measurement in a flow path of the flow cell. The fluorescent particles that emit fluorescence with a known photon energy are fixed. Using a brightness image acquired by imaging fluorescence of the fluorescent particles described above, excitation light irradiation of a sequencer and the operation of a fluorescence detection unit can be checked. Even in measurement of a nucleic acid, a brightness image of fluorescence is used. Therefore, the operation can be checked under the same conditions as the measurement of a nucleic acid.

A blocking layer that is formed of a carboxylic acid compound, a phosphoric acid compound, salt of the compounds, or the like is laminated in the region where only the fluorescent particles are present. Due to the presence of the blocking layer described above, genes (nucleic acids) to be inspected are not adsorbed in the region where only the fluorescent particles are present.

Accordingly, the region where only the fluorescent particles are present and the region where only nucleic acids are present can be formed on the same plane in the flow path. The fluorescent particles can be arranged in the flow path. Therefore, when the fluorescent particles are imaged, the flow path can be filled with a reagent such that the adjacent material of the fluorescent particles to be imaged is the reagent.

That is, when a nucleic acid to be inspected is inspected, the adjacent materials can be made the same. Accordingly, when a brightness image of fluorescence of the fluorescent particles is used for operation check, correction for considering a difference in adjacent material can be made unnecessary.

The region where only the fluorescent particles are present and the region where only a measurement target is present are covered with the blocking layer. Therefore, even when a reaction liquid is supplied, the fluorescent particles and the measurement target do not remove from the fixed surface.

The fluorescent particles are arranged in the vicinity of a liquid flow inlet portion and a liquid flow outlet portion in the flow cell flow path structure. The vicinity of the liquid flow inlet portion and the liquid flow outlet portion are positions where the flow rate of the reaction liquid is not uniform, and are regions that are not used for the measurement. Therefore, the device operation can be checked without reducing the region where the measurement target is arranged.

The invention provides a device for analysis of nucleic acid as a device that acquires an image of a fluorescent material in a flow path using the above-described flow cell, in which a fluorescent image of the fluorescent particles is acquired, the number of fluorescent particles, a distance between particles, a fluorescent image contrast, a fluorescence signal intensity are measured using the acquired image, and whether an operation of the device is normal is determined based on an objective lens position when the fluorescent image is acquired.

The flow cell where the fluorescent particles for operation check and the measurement target are arranged in the same flow path is used. As a result, during the sequence, the device for analysis of nucleic acid can check the operation of the optical system without any intervention of a user such as replacement of the flow cell.

Positions in a height direction of the fluorescent particles for operation check and the measurement target are the same. Therefore, a focusing height of the objective lens for acquiring the fluorescent image of the fluorescent particles for operation check and a focusing height of the objective lens for acquiring the fluorescent image of the measurement target are the same. Therefore, the focusing height of the objective lens can be used as an index for the operation check of the optical system.

Hereinafter, embodiments of the invention will be described with reference to the drawings.

### EMBODIMENTS

### First Embodiment

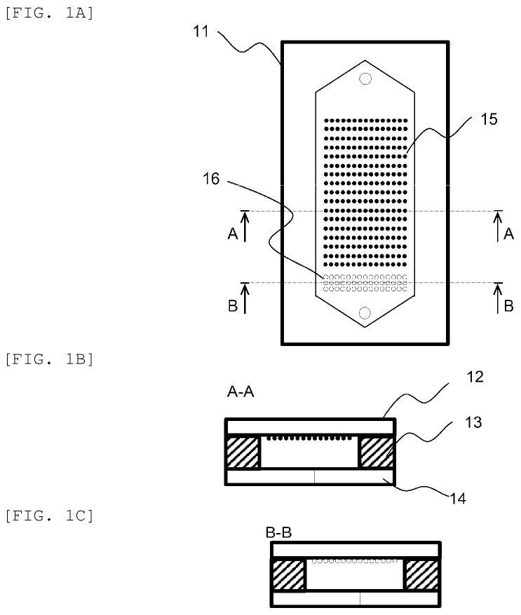

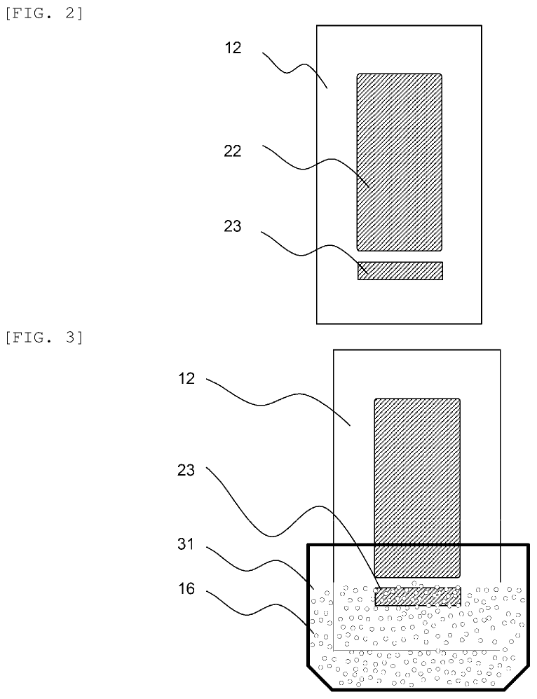

FIG. 1A is a diagram illustrating a flow cell 11 for analysis of nucleic acid according to a first embodiment of the invention. FIG. 1B is a cross-sectional view taken along line A-A of FIG. 1A. FIG. 1C is a cross-sectional view taken along line B-B of FIG. 1A.

The flow cell 11 for analysis of nucleic acid includes a cover plate 12, a spacer 13, and a base plate 14. In the flow cell 11 for analysis of nucleic acid, genes 15 to be inspected and fluorescent particles 16 for device operation check are arranged in a hollow portion. The genes 15 to be inspected and the fluorescent particles 16 for device operation check are fixed on the hollow side of either or both of the cover plate 12 and the base plate 14.

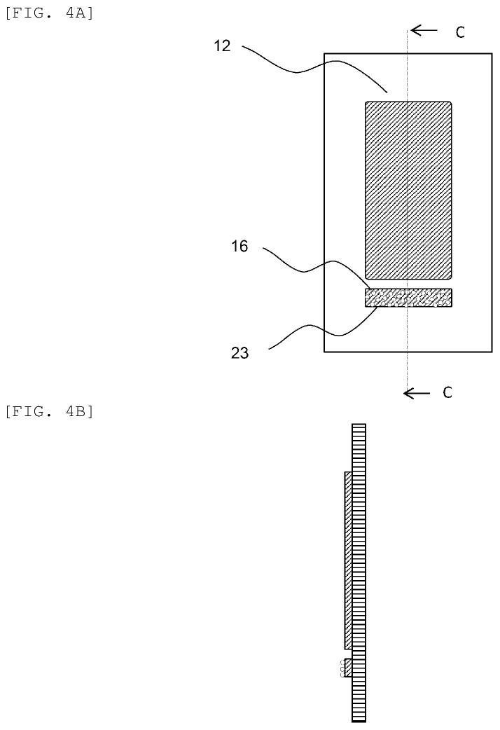

FIG. 2 illustrates a state where a nucleic acid adsorption surface (nucleic acid adsorption region) 22 on which a nucleic acid is adsorbed and a surface 23 on which the fluorescent particles 16 are adsorbed are laminated on the cover plate 12. The cover plate 12 is formed of a material of glass or resin such as acrylic resin through which fluorescence can transmit. On the nucleic acid adsorption surface (nucleic acid adsorption region) 22 and the fluorescent particle adsorption surface (fluorescent particle adsorption region) 23, an inorganic oxide where a functional group including oxygen is known to be specifically adsorbed is deposited by sputtering or vapor deposition. Examples of the inorganic oxide include zirconia and alumina. The nucleic acid adsorption region 22 and the fluorescent particle adsorption region 23 may be formed of the same material. Alternatively, the nucleic acid adsorption region 22 and the fluorescent particle adsorption region 23 may be formed of different materials.

The nucleic acid adsorption region 22 and the adsorption region 23 of the fluorescent particles 16 are separated from each other.

When the flow cell 11 for analysis of nucleic acid is assembled, the region 23 where the fluorescent particles 16 are adsorbed is arranged at a position facing a liquid flow inlet portion (a liquid flow inlet port 81 described below) through which a nucleic acid sample or a reagent flows into a flow path 73 or a liquid flow outlet portion (a liquid flow discharge port 82) through which a nucleic acid sample or reagent flows out from the flow path 73. Specifically, the region 23 is arranged at the position where the flow rate of the reagent is not uniform and that is not used for the measurement of the nucleic acid.

FIG. 3 illustrates a state where the cover plate 12 of FIG. 2 is dipped in a suspension 31 of the fluorescent particles 16. Only the adsorption region 23 of the fluorescent particles 16 is dipped in the suspension. The functional group is bound to the fluorescent particles 16, and the fluorescent particles 16 are fixed in the adsorption region 23 through the functional group. Examples of the functional group include phosphoric acid and carboxylic acid.

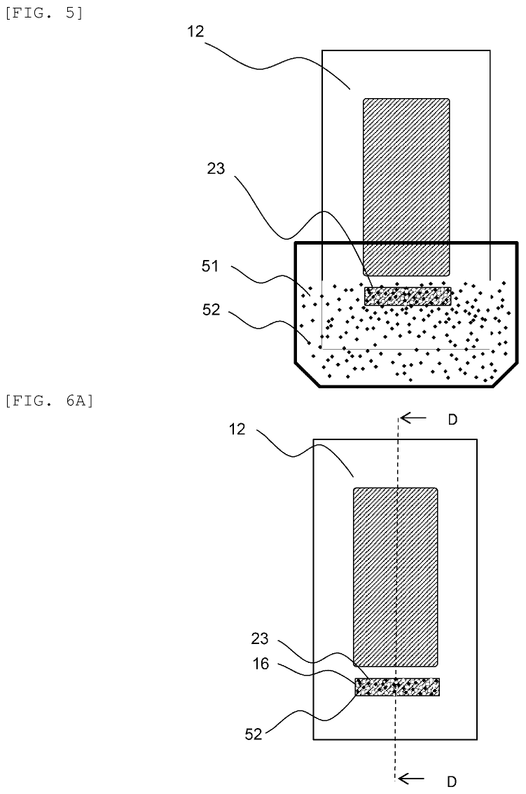

FIG. 4A illustrates a state where the cover plate 12 is pulled up from the suspension illustrated in FIG. 3. FIG. 4B is a cross-sectional view taken along line C-C of FIG. 4A. The fluorescent particles 16 are fixed in the adsorption region 23 of the fluorescent particles 16.

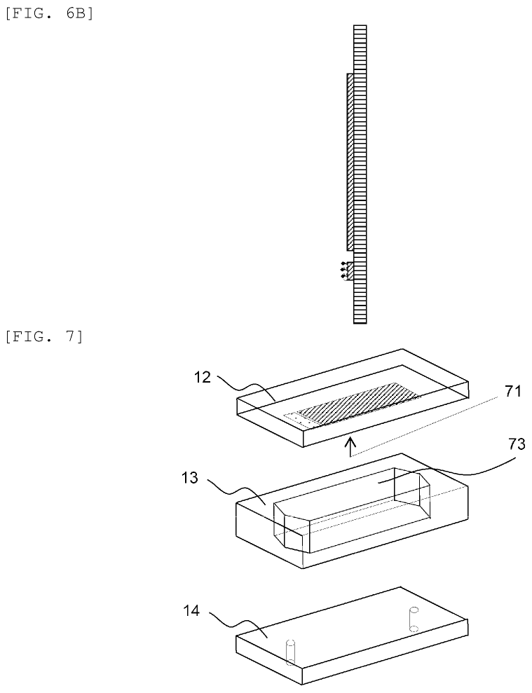

FIG. 5 illustrates a state where the cover plate 12 illustrated in FIG. 4A is dipped in a suspension 51 of a blocking substance 52. The blocking substance 52 is formed of a carboxylic acid compound, a phosphoric acid compound, salt of the compounds, or the like. The compound is specifically adsorbed on the adsorption surface 23 of the fluorescent particles 16.

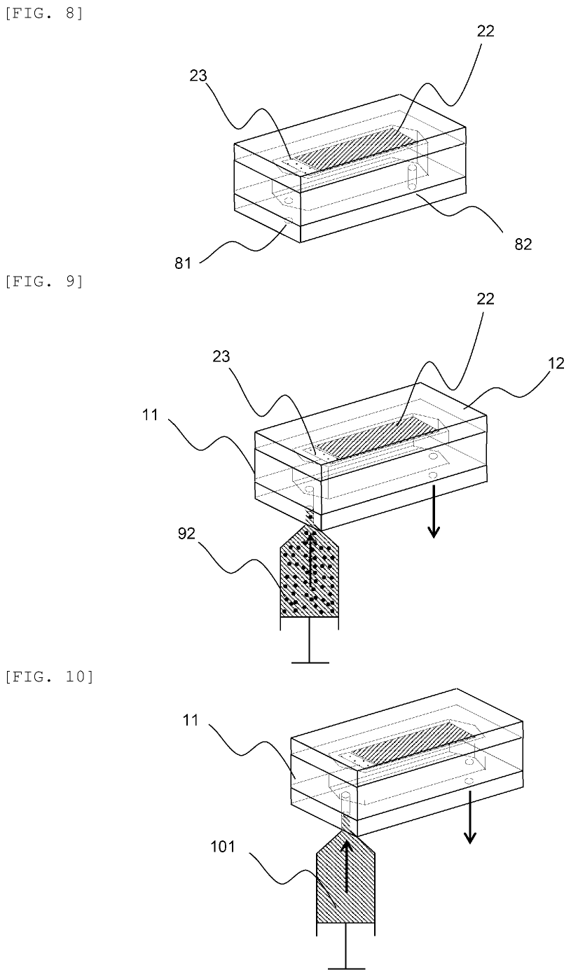

FIG. 6A illustrates a state where the cover plate 12 illustrated in FIG. 5 is pulled up from the suspension of the blocking substance 52. FIG. 6B is a cross-sectional view taken along line D-D of FIG. 6A. In the fluorescent particle adsorption region 23, a region where the fluorescent particles 16 are not adsorbed is covered with the blocking substance 52. Even when the material having phosphoric acid, carboxylic acid, or the like as the functional group is dipped in the portion covered with the blocking substance 52, the material having the functional group such as phosphoric acid or carboxylic acid is not fixed in the portion covered with the blocking substance 52.

FIG. 7 illustrates the configuration of the flow cell using the cover plate 12 illustrated in FIG. 6A. The fluorescent particles 16 are fixed on a fluorescent particle fixed surface 71 of the cover plate 12. The spacer 13 has a structure where the center portion is hollowed out. The spacer 13 is a flow path formation body. During assembly into the flow cell, the hollow portion forms the flow path 73 that is the region through which the liquid passes. For example, the spacer 13 may be a double-sided adhesive tape. The base plate 14 is a plate including two through holes. The two through holes are used as the liquid flow inlet port from the outside of the flow cell and the liquid flow discharge port to the outside of the flow cell. The surface (nucleic acid adsorption region) 22 on which a nucleic acid 92 is adsorbed and the surface (fluorescent particle adsorption region) 23 on which the fluorescent particles 16 are adsorbed that are formed on the cover plate 12 are configured to face the spacer 13 (flow path formation body).

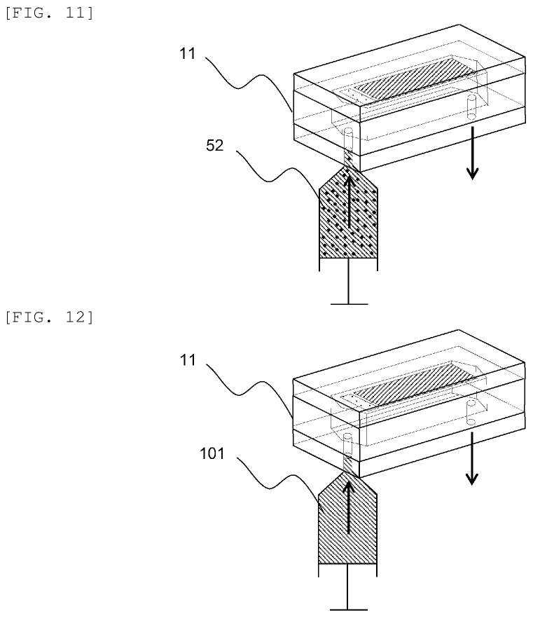

FIG. 8 illustrates a state after the components illustrated in FIG. 7 are assembled. The liquid flowing from the liquid flow inlet port 81 is discharged from the liquid flow discharge port 82. The liquid flow inlet port 81 and the liquid flow discharge port 82 have a structure where, when the liquid passes through a flow path 73 formed between the liquid flow inlet port 81 and the liquid flow discharge port 82, the liquid can come into contact with the nucleic acid adsorption surface 22 and the adsorption region 23 of the fluorescent particles 16.

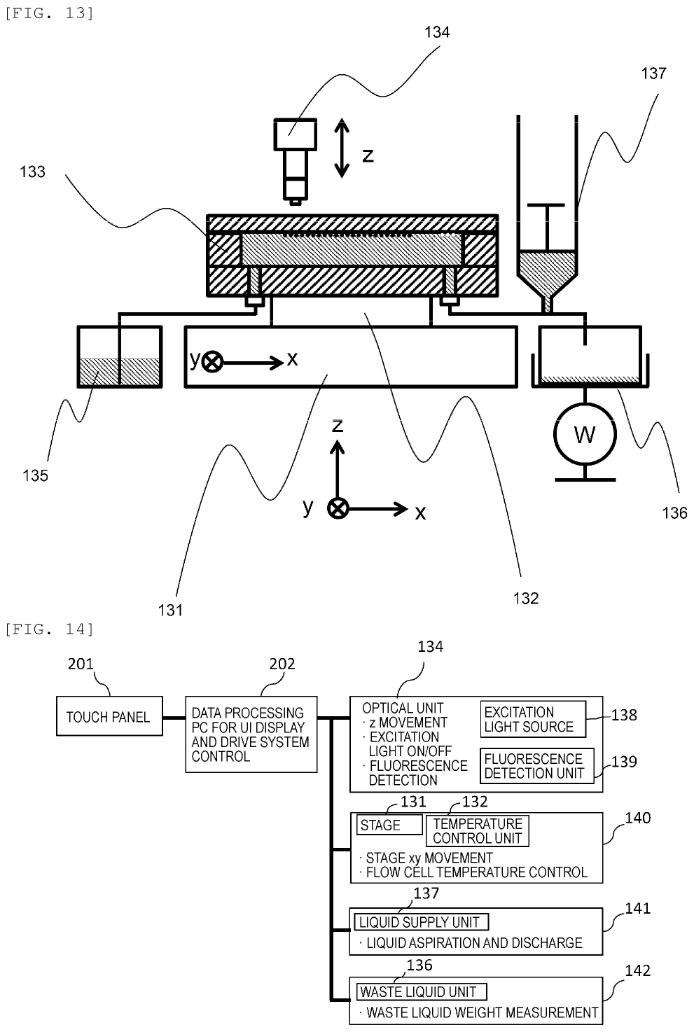

FIG. 9 illustrates a state where a nucleic acid sample liquid including the nucleic acid 92 to be measured is supplied to the flow cell 11 for analysis of nucleic acid illustrated in FIG. 8. The nucleic acid sample to be measured is a nucleic acid of which the base sequence is measured through a sequencing reaction. The functional group is bound to the nucleic acid to be measured, the functional group is adsorbed in the nucleic acid adsorption region 22, and the nucleic acid to be measured is adsorbed in the nucleic acid adsorption region 22 of the cover plate 12 through the functional group. Examples of the functional group include phosphoric acid and carboxylic acid. In the adsorption region 23 of the fluorescent particles 16, the blocking substance 52 is formed, and thus the nucleic acid to be measured is not fixed.

FIG. 10 illustrates a state where a buffer solution 101 is supplied to the flow cell 11 for analysis of nucleic acid of FIG. 9. By supplying the buffer solution 101, the nucleic acid that floats in the flow path without being adsorbed in the region 22 where the nucleic acid is adsorbed is discharged to the outside of the flow cell 11 for analysis of nucleic acid.

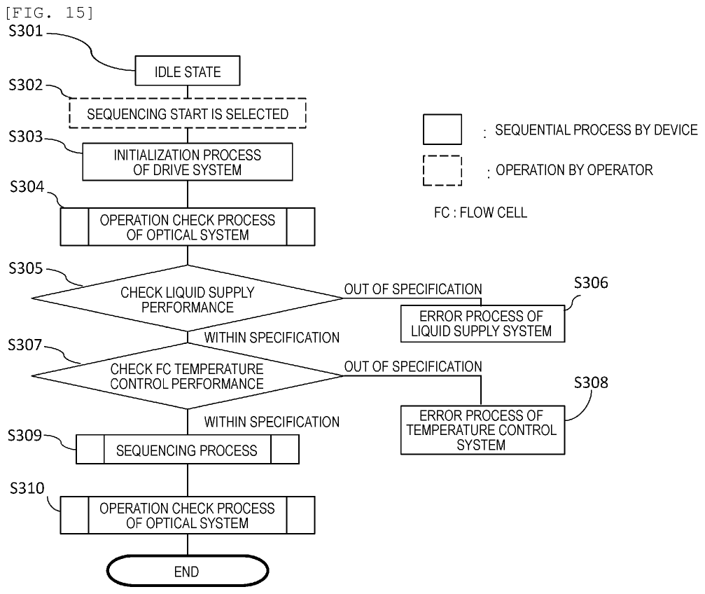

FIG. 11 illustrates a state where the suspension of the blocking substance 52 is supplied to the flow cell 11 for analysis of nucleic acid of FIG. 10. The blocking substance 52 is formed of a carboxylic acid compound, a phosphoric acid compound, or salt thereof. The compound is specifically adsorbed on the fluorescent particle adsorption region 23.

FIG. 12 illustrates a state where the buffer solution 101 is supplied to the flow cell 11 for analysis of nucleic acid of FIG. 11. By supplying the buffer solution 101, the blocking substance 52 that floats in the flow path without being adsorbed in the nucleic acid adsorption region 22 is discharged to the outside of the flow cell 11 for analysis of nucleic acid.

In the operation check of the optical system of the DNA sequencer using the flow cell 11 for analysis of nucleic acid according to the first embodiment of the invention, the excitation light irradiation of the DNA sequencer and the operation of a fluorescence detection unit can be checked using the fluorescent particles 16 arranged at a position that is not used for the measurement, and a decrease in device throughput can be inhibited.

The adjacent materials of the fluorescent particles 16 and the nucleic acid to be measured can be made the same reagent, and signal intensity correction that is required when the adjacent materials are different is unnecessary, and data processing can be simplified.

The sample to be inspected and other materials are separately arranged such that only the sample is arranged in one section and the other material is arranged in another section. As a result, the fluorescent particles 16 can be arranged at a position that is not used for the measurement.

That is, according to the first embodiment of the invention, it is possible to implement the flow cell 11 for analysis of nucleic acid capable of inhibiting complication of data processing without a reduction in throughput of the DNA sequencer.

### Second Embodiment

Next, a second embodiment of the invention will be described.

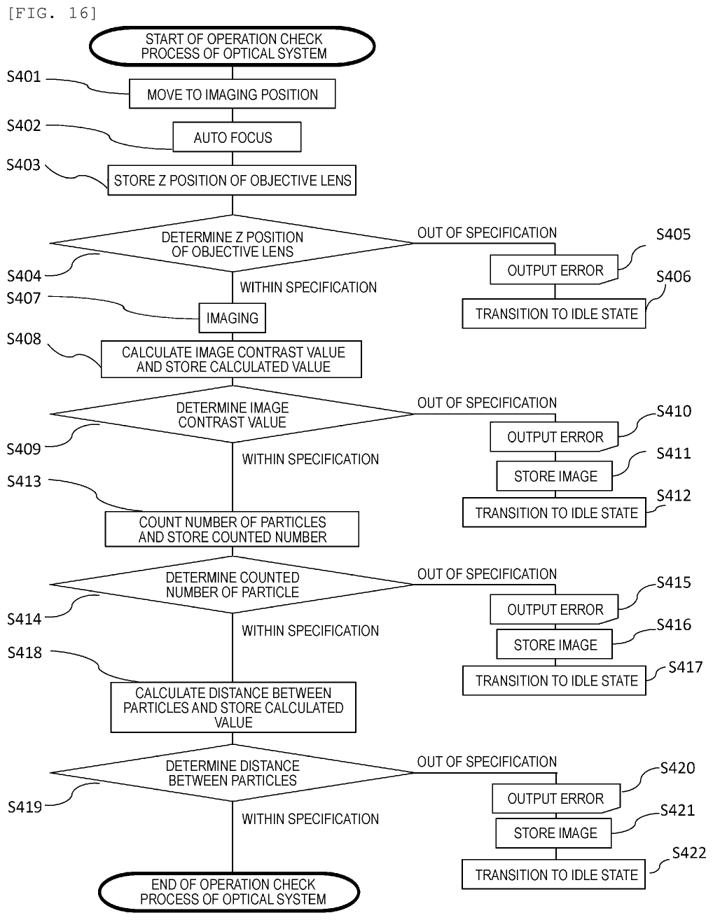

FIG. 13 illustrates a configuration example of a DNA sequencer (device for analysis of nucleic acid) according to the second embodiment. A flow cell 133 for analysis of nucleic acid is arranged in contact with a temperature control unit 132 having a heater function on a stage 131 that moves in an xy direction.

The flow cell 133 for analysis of nucleic acid has the same configuration as the flow cell 11 for analysis of nucleic acid according to the first embodiment.

An excitation light source and fluorescence detection unit 134 is driven in a z direction with respect to the flow cell 133 for analysis of nucleic acid, and a fluorescent label of a nucleic acid and a fluorescent image of fluorescent particles for device operation check are acquired.

The temperature control unit 132 includes a temperature sensor, inspects whether the temperature control of the temperature control unit 132 is within specification, and manages the amount of heat affecting the flow cell 133 for analysis of nucleic acid. In a pipe connected to the flow cell 133 for analysis of nucleic acid, a liquid supply unit 137 that aspirates a reagent and a cleaning solution from a reagent and cleaning liquid tank 135 and discharges the reagent and the cleaning solution to a waste liquid unit 136 is provided. The waste liquid unit 136 includes a weight sensor that measures the weight of a waste solution, inspects whether the weight of the waste solution is within the specification, and manages the supply of the amount of liquid satisfying the specification to the flow cell 133 for analysis of nucleic acid.

FIG. 14 is a flow diagram illustrating a control system of the DNA sequencer. An operator of the DNA sequencer operates the sequencer using a touch panel 201 such that an UI screen is displayed on the touch panel 201 in accordance with the operation. For data processing such as UI display, control of the drive system of the sequencer, and fluorescent image analysis, a data processing PC (data processing control unit configured to perform operation control and fluorescent image analysis) 202 is provided. As the control of the drive system, for example, a z movement of an objective lens of an excitation light source 138 and a fluorescence detection unit 139, excitation light ON/OFF of the excitation light source 138, and a control of fluorescence detection by the fluorescence detection unit 139 are performed. The excitation light source and fluorescence detection unit 134 including the excitation light source 138 and the fluorescence detection unit 139 is provided.

A stage movement and temperature control unit 140 that performs a stage xy movement of the stage 131 and the temperature control unit 132 and a temperature control of the flow cell 133 for analysis of nucleic acid is provided. A liquid supply unit control unit 141 that performs liquid aspiration and discharge of the liquid supply unit 137 is provided. A waste liquid unit control unit 142 that performs waste liquid weight measurement of the waste liquid unit 136 is provided.

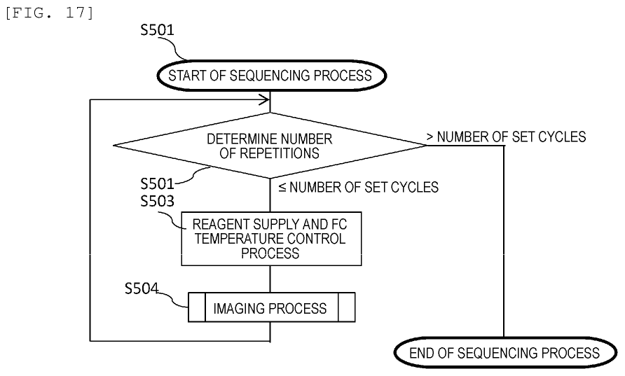

FIG. 15 illustrates an overall process flow of the DNA sequencer. In FIG. 15, a step indicated by a solid line is a sequential process by the device, and a step indicated by a broken line is an operation by the operator.

In the DNA sequencer in an idle state (Step S301), when the sequencing start is selected by the operator (Step S302), the following sequential process is automatically performed by the device.

First, an initializing (return to origin) process of the drive system is performed (Step S303). Next, using the flow cell 133 for analysis of nucleic acid, an operation check process of the optical system of the DNA sequencer is performed (Step S304). When the result of the determination process that is performed in the operation check process of the optical system satisfies the specification, the check of liquid supply performance (Step S305) and the check of flow cell temperature control performance (Step S307) are performed.

In Step S305, to check the liquid supply performance, the weight of the waste liquid is measured using the above-described weight scale, and whether the amount of liquid supplied is within specification is determined. When the result of the liquid supply performance check is out of specification, an error of the liquid supply system is output to the user interface (Step S306), and the sequencing process is not performed in the out-of-specification state.

In Step S307, to check the temperature control performance of the flow cell 133 for analysis of nucleic acid, the temperature of a flow cell contact portion of the temperature control unit 132 is measured using the above-described temperature sensor, and whether the temperature is within specification is determined. When the result of the flow cell temperature control check is out of specification, an error of the temperature control system is output (Step S308), and the sequencing process is not performed in the out-of-specification state.

When all of the operation check results of the optical system, the liquid supply, and the flow cell temperature control are within specification, the sequencing process is performed (Step S309). After the sequencing process, the operation check of the optical system is performed (Step S310). By performing the operation check process of the optical system before and after the sequencing, it is determined that the sequencing is in normal operation.

FIG. 16 illustrates a flowchart illustrating the operation check process (Step S304) of the optical system in FIG. 15.

When the operation inspection process of the optical system starts, for example, the stage 131 on which the flow cell 133 for analysis of nucleic acid is mounted moves to an imaging position (Step S401). The imaging position is the adsorption region 23 of the fluorescent particles 16 that is the device operation check region of the flow cell 133 for analysis of nucleic acid. The fluorescence detection unit 139 in the excitation light source and fluorescence detection unit 134 includes the objective lens and moves the objective lens and the objective lens is auto-focused such that the adsorption region 23 can be imaged through the objective lens (Step S402). When being in focus (when the image of the fluorescent particle adsorption region is acquired), the z position of the objective lens is stored in the data processing PC 202 as the data processing unit (Step S403).

The z position of the objective lens is determined (Step S404), and when the z position is out of specification, an error is output (Step S405), and the process transitions to the idle state (Step S406). In Step S404, when the z position is within specification, an image sensor acquires two-dimensional brightness information (Step S407). Using the two-dimensional brightness information, an image contrast is calculated, and the calculated value is stored in the data processing unit (Step S408).

The determination of the image contrast value is performed (Step S409), and when the image contrast is out of specification, an error is output (Step S410). After storing the image (Step S411), the process transitions to the idle state (Step S412).

In Step S409, when the contrast value is within specification, the number of particles is counted using the acquired image, and the counted number is stored (Step S413). The determination of the counted number is performed (Step S414), and when the count value is out of specification, an error is output (Step S415), and the image is stored (Step S416). Next, the process transitions to the idle state (Step S417).

In Step S414, when the number of particles is within specification, a distance between particles is calculated using the acquired image, and the calculated value is stored (Step S418). The determination of the distance between particles is performed (Step 419), and when the calculated value is out of the specification, an error is output (Step S420), and the image is stored (Step S421). Next, the process transitions to the idle state (Step S422). In Step S419, when the distance between particles is within specification, the operation check of the optical system ends.

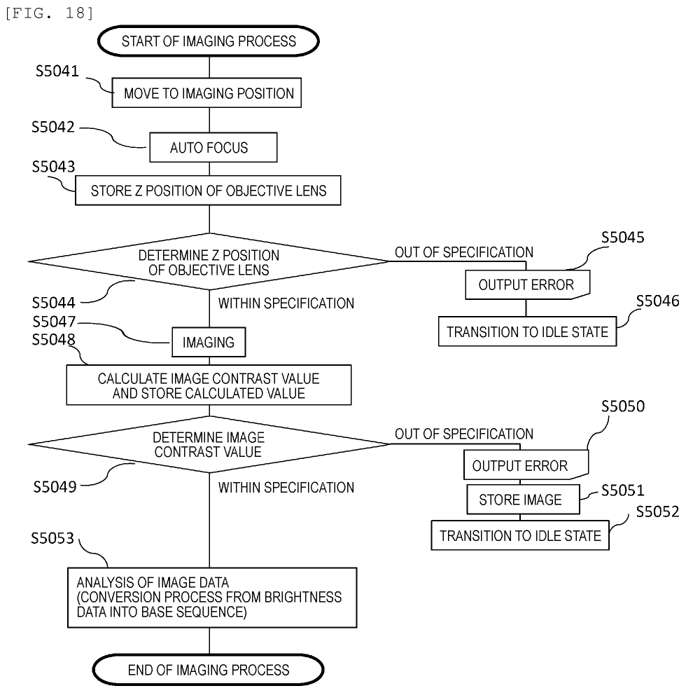

FIG. 17 illustrates a flowchart illustrating the sequencing process (Step S309) in FIG. 15. When the sequencing process starts (Step S501), the reagent supply to the flow cell 133 for analysis of nucleic acid and the flow cell temperature control process (Step S503) and the imaging process (Step S504) are repeated a set number of cycles (Step S501) to identify the base sequence.

FIG. 18 illustrates a flowchart illustrating the imaging process (Step S504) in FIG. 17. When the imaging process starts, for example, the stage 131 on which the flow cell 133 for analysis of nucleic acid is mounted moves to an imaging position (Step S5041). The imaging position is the nucleic acid measurement region (nucleic acid adsorption region 22) of the flow cell 133 for analysis of nucleic acid. The device is auto-focused such that an image can be acquired (Step S5042). When being in focus, the z position of the objective lens is stored in the data processing unit 202 (Step S5043).

Next, the z position of the objective lens is determined (Step S5044), and when the z position is out of specification, an error is output (Step S5045), and the process transitions to the idle state (Step S5046).

In Step S5044, when the z position is within specification, two-dimensional brightness information is acquired as in Step S407 of FIG. 16 (Step S5047). Using the two-dimensional brightness information, an image contrast is calculated, and the calculated value is stored in the data processing unit (Step S5048).

Next, the determination of the image contrast value is performed (Step S5049), and when the image contrast value is out of specification, an error is output (Step S5051). The image is stored (Step S5051). Next, the process transitions to the idle state (Step S5052).

In Step S5049, when the image contrast value is within specification, the image data is analyzed, and a conversion process from the brightness data into the base sequence is performed (Step S5053).

According to the second embodiment of the invention, using fluorescent particles 32 of the flow cell 133 for measurement of nucleic acid, the excitation light irradiation and the operation of a fluorescence detection unit can be checked, and a decrease in device throughput can be inhibited.

The adjacent materials of the fluorescent particles 16 and the nucleic acid to be measured can be made the same reagent, and signal intensity correction that is required when the adjacent materials are different is unnecessary, and data processing can be simplified.

That is, according to the second embodiment of the invention, it is possible to implement the device for analysis of nucleic acid capable of inhibiting complication of data processing without a reduction in throughput of the DNA sequencer.

The cover plate 12 and the base plate 14 can be generally referred to as the flow path formation body support member that supports the flow path formation body (spacer 13) such that the flow path formation body is interposed between the cover plate 12 and the base plate 14. The flow path formation body support member is formed of the cover plate 12 and the base plate 14.

Here, as another embodiment of the invention, a sequencing process method of the device for analysis of nucleic acid is provided.

Provided is a sequencing process method of the device for analysis of nucleic acid that repeats the reagent supply to the flow cell 133 for analysis of nucleic acid, the flow cell temperature control process, and the imaging process a set number of cycles to identify the base sequence, the sequencing process method including: a step of imaging the fluorescent particle adsorption region 23 through the objective lens; a step of analyzing the number of fluorescent particles, a distance between particles, a fluorescent image contrast, and a fluorescence signal intensity using the acquired image of the fluorescent particle adsorption region 23; and a step of determining whether operations of the excitation light source 138 and the fluorescence detection unit 139 are normal depending on a position of the objective lens when the image of the fluorescent particle adsorption region 23 is acquired.

### REFERENCE SIGNS LIST

11, 133: flow cell for analysis of nucleic acid
12: cover plate
13: spacer (flow path formation body)
14: base plate
15: genes to be inspected
16: fluorescent particle
22: surface on which nucleic acid is adsorbed (nucleic acid adsorption region)
23: surface on which fluorescent particles are adsorbed (fluorescent particle adsorption region)
31: suspension of fluorescent particles
51: suspension of blocking substance
52: blocking substance
71: fluorescent particle fixed surface
73: flow path
81: liquid flow inlet port
82: liquid flow discharge port
92: nucleic acid
101: buffer solution
131: stage
132: temperature control unit
134: excitation light source and fluorescence detection unit
135: reagent and cleaning liquid tank
136: waste liquid unit
137: liquid supply unit
138: excitation light source
139: fluorescence detection unit
140: stage movement and temperature control unit
141: liquid supply unit control unit
142: waste liquid unit control unit
201: touch panel
202: data processing PC (data processing control unit configured to perform operation control and fluorescent image analysis)

1. A flow cell for analysis of nucleic acid comprising:
a flow path formation body including a flow path into which a nucleic acid sample flows; and
a flow path formation body support member including a region where a nucleic acid in the nucleic acid sample flowing into the flow path is adsorbed, wherein
the flow path formation body support member includes
a nucleic acid adsorption region where the nucleic acid is adsorbed, and
a fluorescent particle adsorption region that is separated from the nucleic acid adsorption region and where fluorescent particles are adsorbed, and
a blocking substance to be specifically adsorbed on the fluorescent particle adsorption region is adsorbed on the fluorescent particle adsorption region.

2. The flow cell for analysis of nucleic acid according to claim 1, wherein
the flow path formation body support member is formed of a cover plate and a base plate that support the flow path formation body such that the flow path formation body is interposed between the cover plate and the base plate, and
the nucleic acid adsorption region and the fluorescent particle adsorption region are formed on a surface of the cover plate facing the flow path formation body.

3. The flow cell for analysis of nucleic acid according to claim 2, wherein
the blocking substance is a carboxylic acid compound.

4. The flow cell for analysis of nucleic acid according to claim 2, wherein
the blocking substance is a phosphoric acid compound.

5. The flow cell for analysis of nucleic acid according to claim 2, wherein
the cover plate is formed of a material through which fluorescence transmits.

6. The flow cell for analysis of nucleic acid according to claim 2, wherein
an inorganic oxide is deposited on the fluorescent particle adsorption region.

7. The flow cell for analysis of nucleic acid according to claim 2, wherein
a liquid flow inlet port through which the nucleic acid sample flows into the flow path and a liquid flow outlet port through which the nucleic acid sample flows out from the flow path are formed on the base plate and
the fluorescent particle adsorption region is arranged to face the liquid flow inlet port.

8. A device for analysis of nucleic acid comprising:
a flow cell for analysis of nucleic acid;
an excitation light source;
a fluorescence detection unit;
a liquid supply unit; and
a data processing control unit configured to perform operation control and fluorescent image analysis, wherein
the flow cell for analysis of nucleic acid includes
a flow path formation body including a flow path into which a nucleic acid sample flows, and
a flow path formation body support member including a region where a nucleic acid in the nucleic acid sample flowing into the flow path is adsorbed,
the flow path formation body support member includes
a nucleic acid adsorption region where the nucleic acid is adsorbed, and
a fluorescent particle adsorption region that is separated from the nucleic acid adsorption region and where fluorescent particles are adsorbed, and
a blocking substance to be specifically adsorbed on the fluorescent particle adsorption region is adsorbed on the fluorescent particle adsorption region.

9. The device for analysis of nucleic acid according to claim 8, wherein
the fluorescence detection unit includes an objective lens,
the fluorescence detection unit images the fluorescent particle adsorption region through the objective lens, and
the data processing control unit analyzes the number of fluorescent particles, a distance between particles, a fluorescent image contrast, and a fluorescence signal intensity using the acquired image of the fluorescent particle adsorption region, and determines whether operations of the excitation light source and the fluorescence detection unit are normal depending on a position of the objective lens when the image of the fluorescent particle adsorption region is acquired.
